(19) United States
(12) Patent Application Publication
(10) Pub. No.: US 2022/0330934 A1
(43) Pub. Date: Oct. 20, 2022

(54) MINIMALLY INVASIVE SPINAL ANNULUS FIBROSUS REPAIRING DEVICE

(71) Applicants: Yang-Hwei Tsuang, Taipei City (TW); Steve T Lin, Taipei City (TW)

(72) Inventors: Yang-Hwei Tsuang, Taipei City (TW); Fon-Yih Tsuang, Taipei City (TW); Chang-Jung Chiang, Taipei City (TW); Steve T Lin, Taipei City (TW)

(21) Appl. No.: 17/760,656

(22) PCT Filed: Sep. 17, 2020
(86) PCT No.: PCT/US2020/051225

§ 371 Date: Mar. 15, 2022

Related U.S. Application Data
(63) Continuation of application No. 62/901,282, filed on Sep. 17, 2019.

(51) Int. Cl. A61B 17/04; A61F 2/44

(52) U.S. Cl. CPC A61B 17/0401; A61F 2/442; A61B 2017/0414; A61F 2002/4435

(57) ABSTRACT

The present disclosure provides a minimally invasive spinal annulus fibrosus repairing device, including at least one bone anchor, at least one annulus fibrosus anchor, and at least one suture. The minimally invasive spinal annulus fibrosus repairing device of the present disclosure can be used to repair the annulus fibrosus damage, and can be combined with the use of an annulus fibrosus implant to repair the damaged annulus fibrosus.

### CROSS-REFERENCE TO RELATED APPLICATION

This application claims priorities of U.S. patent application No. 62/901,282, filed on Sep. 17, 2019, the content of which is incorporated herein in its entirety by reference.

### BACKGROUND OF THE INVENTION

### 1. Field of the Invention

The present invention relates to a minimally invasive spinal annulus fibrosus repairing device.

### 2. The Prior Art

The intervertebral disc, located between the adjacent vertebrae of the spine, is a very specialized and highly organized fibrocartilage structure. Without the structure of the intervertebral disc, the spine will not be able to support load or maintain elasticity. In a healthy intervertebral disc, the nucleus pulposus (NP) in the central part can maintain and transmit load, and the annulus fibrosus (AF) around the nucleus pulposus can provide compression and mobility of the spine.

Defects of the annulus fibrosus, such as peripheral peeling and radial cracks, may cause squeezing of the nucleus pulposus tissue, which is called a herniated disc. Especially on the back side of the spine, the herniated disc or a fragmented nucleus pulposus can cause pain due to compression of the nerve root. There are many different methods for treating the herniated disc, among which discectomy (such as nucleus pulposus removal) is a standard solution today. According to research, the use of intervertebral discectomy and repair of the annulus fibrosus defect treatment can enhance clinical technology and efficacy.

In the past, surgeons tried to use standard sutures, stitches, and microscopy techniques to close the intervertebral disc and its annulus fibrosus tears. However, due to the limitations of microdiscectomy, standard suture techniques are difficult to implement.

In addition, conventionally, it is necessary to draw the suture when suturing the tear of the annulus fibrosus. Generally, an included angle of 0 degree is formed between the pulling direction of the suture and an extension direction that is far away from and perpendicular to a tear of the annulus fibrosus of the intervertebral disc. However, the force generated by this pulling direction is relatively large, which may cause the suture to separate from the tear or pull out the repairing device placed in the tear of the annulus fibrosus. On the contrary, the wound will be torn again, causing secondary injury.

In order to solve the above problems, those skilled in the art urgently need to develop a minimally invasive spinal annulus fibrosus repairing device for the benefit of a large group of people in need thereof.

### SUMMARY OF THE INVENTION

A primary objective of the present invention is to provide a minimally invasive spinal annulus fibrosus repairing device, comprising at least one bone anchor, at least one annulus fibrosus anchor, and at least one suture. The at least one bone anchor is configured to be vertically disposed on a posterior surface of a vertebral bone adjacent to an intervertebral disc, wherein the at least one bone anchor includes a body with a needle tip and a suture fixing portion away from the needle tip, the suture fixing portion forms a perforation that penetrates the body radially, and the body is made of a porous structure. The at least one annulus fibrosus anchor is configured to be vertically disposed on the annulus fibrosus of the intervertebral disc, wherein the at least one annulus fibrosus anchor has a first surface and a second surface spaced apart from the first surface, at least two suture inlet holes are formed on the first surface, at least one suture outlet hole is formed on the second surface, and a suture penetrating tunnel is defined between the at least two suture inlet holes and the at least one suture outlet hole. The at least one suture has a first end and a second end located at an opposite end of the first end, wherein the first end and the second end are threaded in through corresponding suture inlet holes respectively, pass through the suture penetrating tunnel, and exit from the at least one suture outlet hole, and the first end and the second end of the at least one suture are used to be fixed to the suture fixing portion of the at least one bone anchor.

According to an embodiment of the present invention, when an amount of the bone anchor is one, an amount of the annulus fibrosus anchor is at least two, when the amount of the annulus fibrosus anchor is one, the amount of the bone anchor is at least two, the at least one bone anchor and the at least one annulus fibrosus anchor define a repairing region, and the repairing region has an area larger than that of a tear of the annulus fibrosus of the intervertebral disc.

According to an embodiment of the present invention, the amount of the at least one bone anchor is two, and the amount of the at least one annulus fibrosus anchor is two.

According to an embodiment of the present invention, when the amount of the bone anchor and the amount of the annulus fibrosus anchor are at least two each, the amount of the bone anchor and the amount of the annulus fibrosis anchor may be the same or different.

According to an embodiment of the present invention, the at least one suture forms a pulling direction after exiting from the at least one suture outlet hole, and an included angle of 90 degrees is formed between the pulling direction and an extension direction that is far away from and perpendicular to a tear of the annulus fibrosus of the intervertebral disc.

According to an embodiment of the present invention, the at least one bone anchor has a length of 10-20 mm and a width of 3-5 mm.

According to an embodiment of the present invention, the at least one annulus fibrosus anchor has a length of 3-6 mm and a width of 1-3 mm.

According to an embodiment of the present invention, an amount of the suture inlet holes is two.

According to an embodiment of the present invention, the suture penetrating tunnel includes a first tunnel portion and a second tunnel portion opposite to the first tunnel portion, and an included angle of 30-90 degrees is formed between an extension line of the first tunnel portion and an extension line of the second tunnel portion.

According to an embodiment of the present invention, the at least one annulus fibrosus anchor has a columnar structure.

According to an embodiment of the present invention, the at least one annulus fibrosus anchor has a cylindrical structure.

According to an embodiment of the present invention, the at least one annulus fibrosus anchor has a material made of artificial fiber, heterogeneous fiber, titanium alloy, cobalt-based alloy, ceramic, stainless steel, polyetheretherketone (PEEK) resin, or artificial bone.

According to an embodiment of the present invention, the first end and the second end of the at least one suture pass through the suture penetrating tunnel and exit from the at least one suture outlet hole, and the first end and the second end of the at least one suture fixed on the suture fixing portion of the at least one bone anchor are drawn to form a bundle shape in order to seal a tear of the fibrous annulus of the intervertebral disc.

According to an embodiment of the present invention, the body of the at least one bone anchor has a cross-sectional shape of polygon or sheet.

According to an embodiment of the present invention, the suture fixing portion of the body of the at least one bone anchor is a loop.

According to an embodiment of the present invention, the body of the at least one bone anchor further includes a solid frame, and the porous core structure is concentrated inside the solid frame and presented in a center of the solid frame.

According to an embodiment of the present invention, the at least one bone anchor has a shape of a porous anchor, a quadrangular prismatic anchor, a threaded anchor, a staple or a nail.

Another objective of the present invention is to provide a method of using the above mentioned minimally invasive spinal annulus fibrosus repairing device, comprising the following steps: fixing the at least one bone anchor on the vertebral bone adjacent to the intervertebral disc, wherein the needle tip is embedded in the vertebral bone, and the suture fixing portion is exposed out of the vertebral bone; threading the first end and the second end of the at least one suture through the at least two suture inlet holes of the at least one annulus fibrosus anchor, and passing through the suture penetrating tunnel to exit from the at least one suture outlet hole; implanting the at least one annulus fibrosus anchor with the at least one suture into the annulus fibrosus of the intervertebral disc, and pulling the first end and the second end of the at least one suture out of the annulus fibrosus of the intervertebral disc; and spanning the at least one suture that is pulled out of the annulus fibrosus of the intervertebral disc over a tear of the annulus fibrosus of the intervertebral disc, wherein the first end and the second end of the at least one suture are fixed to the suture fixing portion of the at least one bone anchor.

In summary, the minimally invasive spinal annulus fibrosus repairing device of the present invention has the following effect: by the arrangement that at least two suture inlet holes are formed on the first surface, at least one suture outlet hole is formed on the second surface, a suture penetrating tunnel is defined between the at least two suture inlet holes and the at least one suture outlet hole, the first end and the second end are threaded in through corresponding suture inlet holes respectively, pass through the suture penetrating tunnel, and exit from the at least one suture outlet hole, and the suture is slidable relative to the annulus fibrosus anchor, the suture can slide freely relative to the annulus fibrosus anchor without being tied up. The first end and the second end of the suture pass through the suture penetrating tunnel and exit from the at least one suture outlet hole, and the first end and the second end of the at least one suture fixed on the suture fixing portion of the at least one bone anchor are drawn to form a bundle shape in order to seal a tear of the fibrous annulus of the intervertebral disc and reduce the risk of reoperation. In addition, with the configuration made of the porous structure of the body of the bone anchor, the vertebral bone can be extended into the porous structure as it grows, and the bone anchor can be stably positioned on the vertebral bone, making the bone anchor not easy to slip off. In addition, an included angle of 90 degrees is formed between the pulling direction of the suture and an extension direction that is far away from and perpendicular to a tear of the annulus fibrosus of the intervertebral disc. The force generated by the pulling direction is relatively small, the suture will not be separated from the tear, the repairing device placed in the tear of the annulus fibrosus will not be pulled out, and the wound will not be torn again, causing secondary injury. The minimally invasive spinal annulus fibrosus repairing device of the present invention can be used to repair annulus fibrosus damage, and can be combined with the use of annulus fibrosus implants to repair tears in the annulus fibrosus.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 is a schematic diagram of the intervertebral disc and the annulus fibrosis.

FIG. 2 is a schematic diagram showing an embodiment of the minimally invasive spinal annulus fibrosus repairing device of the present invention.

FIG. 3 is another schematic diagram showing an embodiment of the minimally invasive spinal annulus fibrosus repairing device of the present invention.

FIG. 4 is a schematic diagram showing an embodiment of the bone fixation anchor.

FIG. 5 is a schematic diagram showing an embodiment of the annulus fibrosus anchor and the suture.

FIG. 6 is a schematic diagram of the suture method.

FIGS. 7A and 7B are photographs showing an embodiment of the minimally invasive spinal annulus fibrosus repairing device of the present invention.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

As used herein, the data provided represent experimental values that can vary within a range of ±20%, preferably within ±10%, and most preferably within ±5%.

Hereinafter, embodiments of the minimally invasive spinal annulus fibrosus repairing device according to the present invention will be described with reference to related drawings, in which the same components will be described with the same reference numbers.

The minimally invasive spinal annulus fibrosus repairing device of the present invention has the following effect: by the arrangement that at least two suture inlet holes are formed on the first surface, at least one suture outlet hole is formed on the second surface, a suture penetrating tunnel is defined between the at least two suture inlet holes and the at least one suture outlet hole, the first end and the second end are threaded in through corresponding suture inlet holes respectively, pass through the suture penetrating tunnel, and exit from the at least one suture outlet hole, and the suture is slidable relative to the annulus fibrosus anchor, the suture can slide freely relative to the annulus fibrosus anchor without being tied up. The first end and the second end of the suture pass through the suture penetrating tunnel and exit from the at least one suture outlet hole, and the first end and the second end of the at least one suture fixed on the suture fixing portion of the at least one bone anchor are drawn to form a bundle shape in order to seal a tear of the fibrous annulus of the intervertebral disc and reduce the risk of reoperation. In addition, with the configuration made of the porous structure of the body of the bone anchor, the vertebral bone can be extended into the porous structure as it grows, and the bone anchor can be stably positioned on the vertebral bone, making the bone anchor not easy to slip off. In addition, an included angle of 90 degrees is formed between the pulling direction of the suture and an extension direction that is far away from and perpendicular to a tear of the annulus fibrosus of the intervertebral disc. The force generated by the pulling direction is relatively small, the suture will not be separated from the tear, the repairing device placed in the tear of the annulus fibrosus will not be pulled out, and the wound will not be torn again, causing secondary injury. The minimally invasive spinal annulus fibrosus repairing device of the present invention can be used to repair annulus fibrosus damage, and can be combined with the use of annulus fibrosus implants to repair tears in the annulus fibrosus. The following examples are used to illustrate the structure and features of the minimally invasive spinal annulus fibrosus repairing device of the present invention. Various combination of various amount of bone anchor and annulus fibrosus anchors can be used for variety of annulus defects. Although standard combination is two bone anchors and two annulus anchors. The suture pass through these anchors form a loop that can be tightened up to make anchors close together.

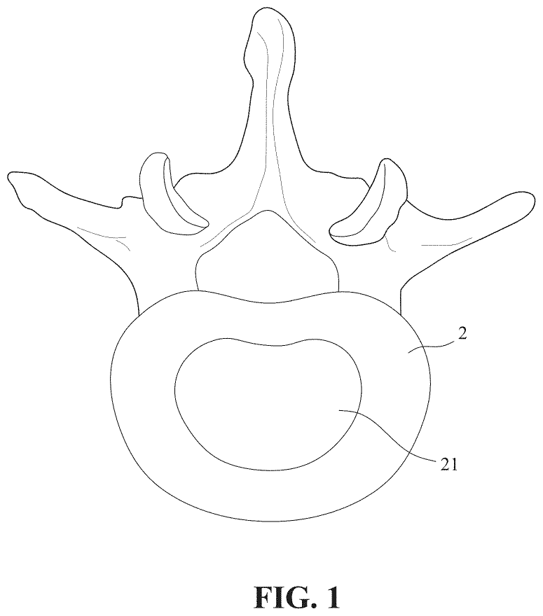

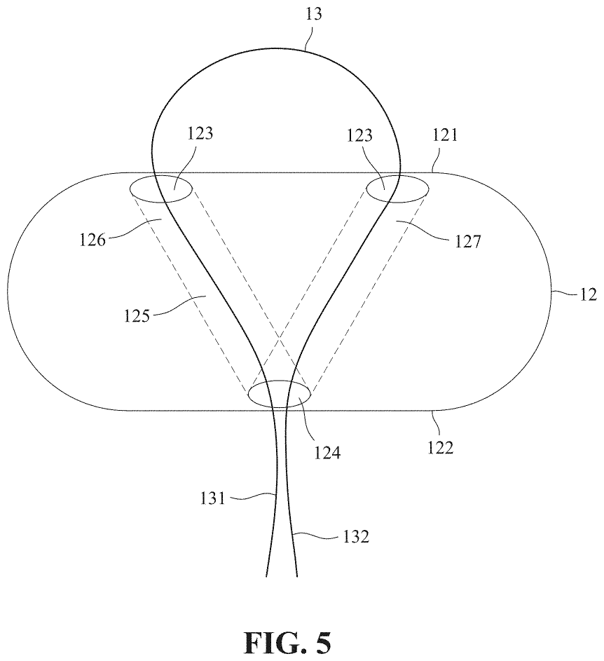

Referring to FIGS. 1 to 5, which are schematic diagrams of an embodiment of the minimally invasive spinal annulus fibrosus repairing device 1 of the present invention, in which FIG. 1 is a schematic diagram of the intervertebral disc 2 and the annulus fibrosus 21. The minimally invasive spinal annulus fibrosus repairing device 1 comprises at least one bone anchor 11, at least one annulus fibrosus anchor 12, and at least one suture 13. The at least one bone anchor 11 is configured to be vertically disposed on a posterior surface of a vertebral bone adjacent to the intervertebral disc 2, wherein the at least one bone anchor 11 includes a body 111 with a needle tip 112 and a suture fixing portion 114 away from the needle tip 112, the suture fixing portion 114 forms a perforation that penetrates the body 111 radially, and the body 111 is made of a porous structure. The at least one annulus fibrosus anchor 12 is configured to be vertically disposed on the annulus fibrosus 21 of the intervertebral disc 2, wherein the at least one annulus fibrosus anchor 12 has a first surface 121 and a second surface 122 spaced apart from the first surface 121, at least two suture inlet holes 123 are formed on the first surface 121, at least one suture outlet hole 124 is formed on the second surface 122, and a suture penetrating tunnel 125 is defined between the at least two suture inlet holes 123 and the at least one suture outlet hole 124. The at least one suture 13 has a first end 131 and a second end 132 located at an opposite end of the first end 131, wherein the first end 131 and the second end 132 are threaded in through corresponding suture inlet holes 123 respectively, pass through the suture penetrating tunnel 125, and exit from the at least one suture outlet hole 124, and the first end 131 and the second end 132 of the at least one suture 13 are used to be fixed to the suture fixing portion 114 of the at least one bone anchor 11.

An embodiment of the present invention provides a method of using the above mentioned minimally invasive spinal annulus fibrosus repairing device, comprising the following steps: fixing the at least one bone anchor 11 on the vertebral bone of the annulus fibrous 21 adjacent to the intervertebral disc 2, wherein the needle tip 112 is embedded in the vertebral bone, and the suture fixing portion 114 is exposed out of the vertebral bone; threading the first end 131 and the second end 132 of the at least one suture 13 through the at least two suture inlet holes 123 of the at least one annulus fibrosus anchor 12, and passing through the suture penetrating tunnel 125 to exit from the at least one suture outlet hole 124; implanting the at least one annulus fibrosus anchor 12 with the at least one suture 13 into the annulus fibrosus 21 of the intervertebral disc 2, and pulling the first end 131 and the second end 132 of the at least one suture 13 out of the annulus fibrosus 21 of the intervertebral disc 2; and spanning the at least one suture 13 that is pulled out of the annulus fibrosus 21 of the intervertebral disc 2 over a tear 22 of the annulus fibrosus 21 of the intervertebral disc 2, wherein the first end 131 and the second end 132 of the at least one suture 13 are fixed to the suture fixing portion 114 of the at least one bone anchor 11.

In the present example, when an amount of the bone anchor 11 is one, an amount of the annulus fibrosus anchor 12 is at least two, when the amount of the annulus fibrosus anchor 12 is one, the amount of the bone anchor 11 is at least two, the at least one bone anchor 11 and the at least one annulus fibrosus anchor 12 define a repairing region 14, and the repairing region 14 has an area larger than that of a tear 22 of the annulus fibrosus 21 of the intervertebral disc 2.

In the present example, the amount of the at least one bone anchor 11 is two, and the amount of the at least one annulus fibrosus anchor 12 is two.

In the present example, when the amount of the bone anchor 11 and the amount of the annulus fibrosus anchor 12 are at least two each, the amount of the bone anchor 11 and the amount of the annulus fibrosis anchor 12 may be the same or different.

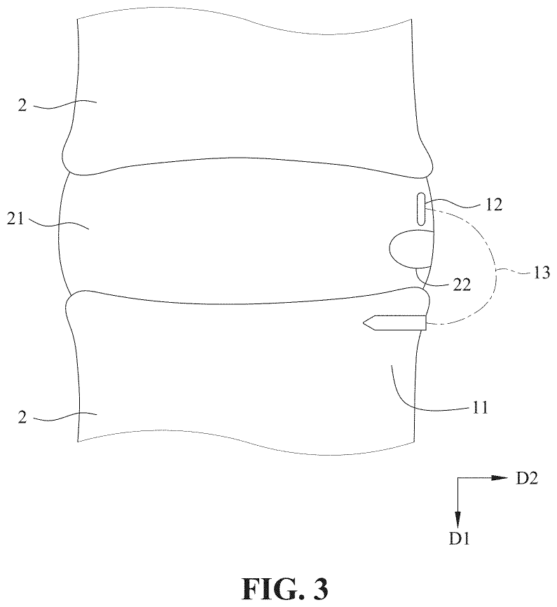

In the present example, the at least one suture 13 forms a pulling direction D1 after exiting from the at least one suture outlet hole 124, and an included angle of 90 degrees is formed between the pulling direction D1 and an extension direction D2 that is far away from and perpendicular to a tear 22 of the annulus fibrosus 21 of the intervertebral disc 2 (see FIG. 3).

Conventionally, it is necessary to draw the suture when suturing the tear of the annulus fibrosus. Generally, an included angle of 0 degree is formed between the pulling direction of the suture and an extension direction that is far away from and perpendicular to a tear of the annulus fibrosus of the intervertebral disc. However, the force generated by this pulling direction is relatively large, which may cause the suture to separate from the tear or pull out the repairing device placed in the tear of the annulus fibrosus. On the contrary, the wound will be torn again, causing secondary injury. On the contrary, the included angle of 90 degrees is formed between the pulling direction D1 and the extension direction D2 that is far away from and perpendicular to the tear 22 of the annulus fibrosus 21 of the intervertebral disc 2. Table 1 shows the force (N) generated by the bone anchor 11 in the direction of pulling the suture at different angles. Table 2 shows the average (AVG) force (N) generated by the annulus fibrosus anchor 12 in the direction of pulling the suture at different angles. The bone anchor 11 used is made of titanium alloy 3×3×10 mm, the annulus fibrosus anchor 12 used is made of polyetheretherketone (PEEK) resin, the outside diameter (OD) is 2 mm, and the length is 3 mm, and the suture 13 used is made of nylon. From Table 1 and Table 2, it can be seen that the average force generated by the included angle of 90 degrees would be less than the average force generated by the included angle of 0 degree.

TABLE 1

| | 0° | 45° | 90° |
|---|---|---|---|
| AVG | 21.5 ± 0.8 | 21.0 ± 1.6 | 19.8.5 ± 0.7 |
| | (N) | (N) | (N) |

TABLE 2

| | 0° | 45° | 90° |
|---|---|---|---|
| AVG | 18.5 ± 3.4 | 12.2 ± 1.2 | 12.6 ± 6.7 |
| | (N) | (N) | (N) |

In the present example, the at least one bone anchor 11 has a length of 10-20 mm and a width of 3-5 mm Preferably, the at least one bone anchor 11 has a length of 10 mm and a width of 3 mm.

In the present example, the at least one annulus fibrosus anchor 12 has a length of 3-5 mm and a width of 1-3 mm Preferably, the at least one annulus fibrosus anchor 12 has a length of 3.5 mm and a width of 1.5 mm.

In the present example, an amount of the suture inlet holes 123 is two.

In the present example, the suture penetrating tunnel 125 includes a first tunnel portion 126 and a second tunnel portion 127 opposite to the first tunnel portion 126, and an included angle of 30-90 degrees is formed between an extension line of the first tunnel portion 126 and an extension line of the second tunnel portion 127. Preferably, an included angle of 60 degrees is formed between the extension line of the first tunnel portion 126 and the extension line of the second tunnel portion 127.

In the present example, the at least one annulus fibrosus anchor 12 has a columnar structure. Preferably, the at least one annulus fibrosus anchor 12 has a cylindrical structure.

In the present example, the at least one annulus fibrosus anchor 12 has a material made of artificial fiber, heterogeneous fiber, titanium alloy, cobalt-based alloy, ceramic, stainless steel, polyetheretherketone (PEEK) resin, or artificial bone.

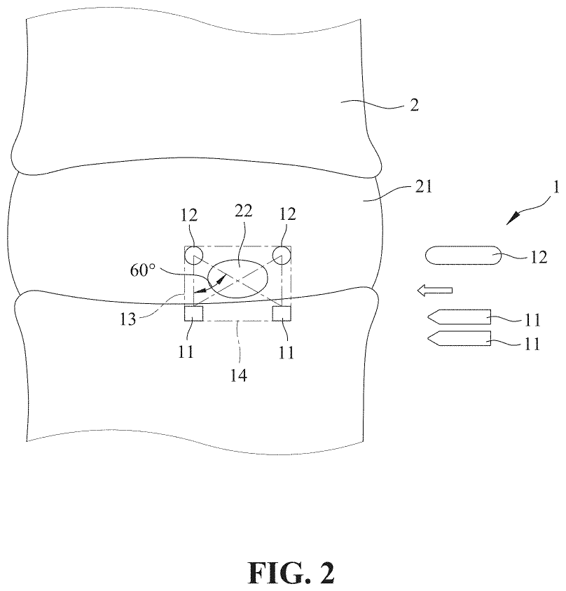

In the present example, the first end 131 and the second end 132 of the at least one suture 13 pass through the suture penetrating tunnel 125 and exit from the at least one suture outlet hole 124, and the first end 131 and the second end 132 of the at least one suture 13 fixed on the suture fixing portion 114 of the at least one bone anchor 11 are drawn to form a bundle shape in order to seal a tear 22 of the fibrous annulus 21 of the intervertebral disc 2. In other words, the annulus fibrosus anchor 12 has a multi-hole cylindrical structure (two suture inlet holes 123 and one suture outlet hole 124), and the suture 13 is threaded through the annulus fibrosus anchor 12. A hollow needle is used to puncture around the damaged part of the fibrous annulus 21, and guide the suture 13 and the annulus fibrosus anchor 12 to the inside of the fibrous annulus 21 through the hollow needle. After removing the hollow needle, the suture 13 is tightened to make the annulus fibrosus anchor 12 be fixed inside the fibrous annulus 21 (see FIG. 2). The two annulus fibrosus anchors 12 and the suture 13 must form an angle of about 60 degrees with the position of the bone anchor 11. The suture 13 squeezes the tear of the fibrous annulus and makes the tear tightly closed. The suture 13 is located between the bone anchor 11 and the annulus fibrosus anchor 12. It is a dynamic fixation method. The suture 13 is not fixed at a specific point, so that when the shape of the spine changes due to the force of the spine, each part of the suture 13 can maintain the same suture stress.

In the present example, the body 111 of the at least one bone anchor 11 has a cross-sectional shape of polygon or sheet.

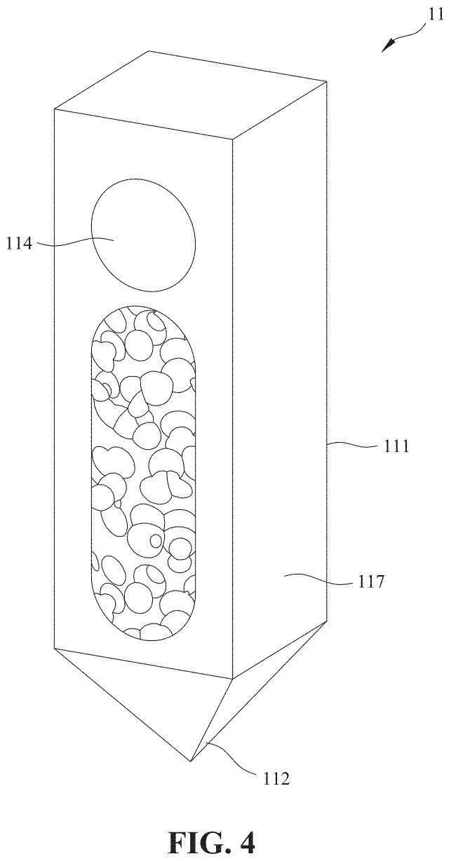

In the present example, the suture fixing portion 114 of the body 111 of the at least one bone anchor 11 is a loop (see FIG. 4).

In the present example, the body 111 of the at least one bone anchor 11 further includes a solid frame 117, and the porous structure is concentrated inside the solid frame 117 and presented in a center of the solid frame 117 (see FIG. 4).

In the present example, the at least one bone anchor 11 has a shape of a porous anchor, a quadrangular prismatic anchor (see FIG. 4), a threaded anchor or a nail.

The bone anchor 11 of the present invention is connected with the suture 13 and can be fixed to the vertebral bone of the intervertebral disc 2. No matter the shape of the bone anchor 11 is a porous anchor, a quadrangular prismatic anchor, a threaded anchor or a nail, it can achieve immediate and strong fixation, and the fixation strength can be maintained for a period of time, which is sufficient for the healing of the annulus fibrosus injury. The size of the porous anchor is a polygonal column or cylinder with a length of 10-20 mm and a width of 3-5 mm. The bone anchor 11 of the quadrangular prismatic anchor has a cylindrical core with the same side length as a porous structure. The proximal end of the threaded anchor has no thread.

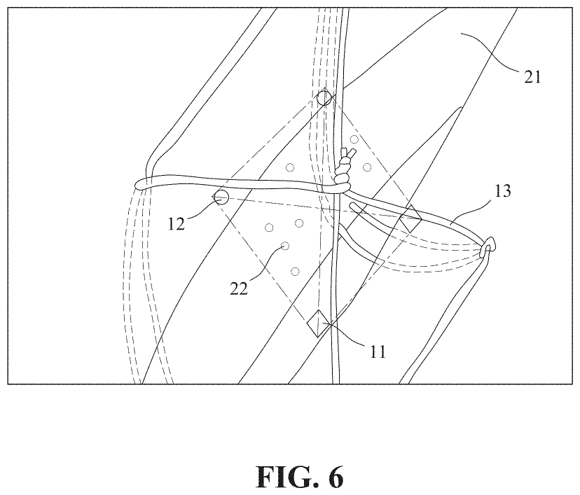

The suturing method of the suture 13 is explained below. The suture 13 is used to suture the tear 22 of the annulus fibrosus 21. The suture 13 is sutured in a purse-string suture method to achieve a water-tight degree to tighten the tear 22 of the annulus fibrosus 21, as shown in FIG. 6.

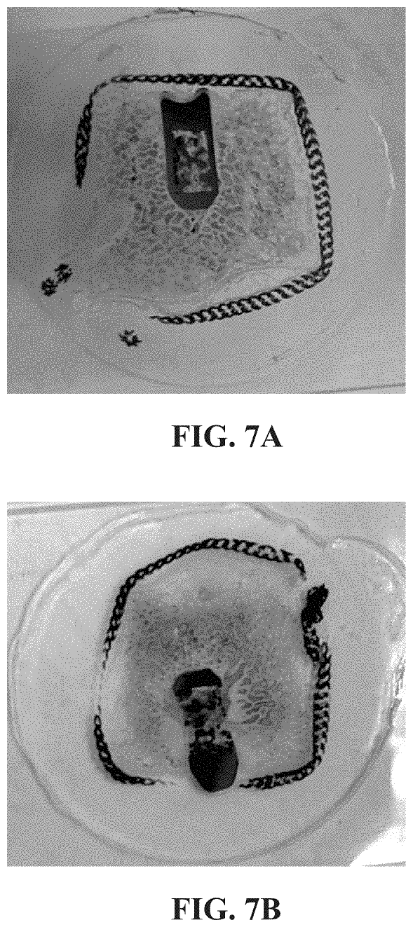

FIGS. 7A and 7B are photographs showing an embodiment of the minimally invasive spinal annulus fibrosus repairing device of the present invention. It can be seen from FIG. 7A and FIG. 7B that the slice observation of the lumbar vertebrae of pigs 3 months after the implantation of the bone anchor. The bones turn brown after staining and it is shown that the bones are grown into the holes in the bone anchor.

In summary, the minimally invasive spinal annulus fibrosus repairing device of the present invention has the following effect: by the arrangement that at least two suture inlet holes are formed on the first surface, at least one suture outlet hole is formed on the second surface, a suture penetrating tunnel is defined between the at least two suture inlet holes and the at least one suture outlet hole, the first end and the second end are threaded in through corresponding suture inlet holes respectively, pass through the suture penetrating tunnel, and exit from the at least one suture outlet hole, and the suture is slidable relative to the annulus fibrosus anchor, the suture can slide freely relative to the annulus fibrosus anchor without being tied up. The first end and the second end of the suture pass through the suture penetrating tunnel and exit from the at least one suture outlet hole, and the first end and the second end of the at least one suture fixed on the suture fixing portion of the at least one bone anchor are drawn to form a bundle shape in order to seal a tear of the fibrous annulus of the intervertebral disc and reduce the risk of reoperation. In addition, with the configuration made of the porous structure of the body of the bone anchor, the vertebral bone can be extended into the porous structure as it grows, and the bone anchor can be stably positioned on the vertebral bone, making the bone anchor not easy to slip off. In addition, an included angle of 90 degrees is formed between the pulling direction of the suture and an extension direction that is far away from and perpendicular to a tear of the annulus fibrosus of the intervertebral disc. The force generated by the pulling direction is relatively small, the suture will not be separated from the tear, the repairing device placed in the tear of the annulus fibrosus will not be pulled out, and the wound will not be torn again, causing secondary injury. The minimally invasive spinal annulus fibrosus repairing device of the present invention can be used to repair annulus fibrosus damage, and can be combined with the use of annulus fibrosus implants to repair tears in the annulus fibrosus.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A minimally invasive spinal annulus fibrosus repairing device, comprising:
at least one bone anchor, configured to be vertically disposed on a posterior surface of a vertebral bone adjacent to an intervertebral disc, wherein the at least one bone anchor includes a body with a needle tip and a suture fixing portion away from the needle tip, the suture fixing portion forms a perforation that penetrates the body radially, and the body is made of a porous structure;
at least one annulus fibrosus anchor, configured to be vertically disposed on the annulus fibrosus of the intervertebral disc, wherein the at least one annulus fibrosus anchor has a first surface and a second surface spaced apart from the first surface, at least two suture inlet holes are formed on the first surface, at least one suture outlet hole is formed on the second surface, and a suture penetrating tunnel is defined between the at least two suture inlet holes and the at least one suture outlet hole; and
at least one suture, having a first end and a second end located at an opposite end of the first end, wherein the first end and the second end are threaded in through corresponding suture inlet holes respectively, pass through the suture penetrating tunnel, and exit from the at least one suture outlet hole, and the first end and the second end of the at least one suture are used to be fixed to the suture fixing portion of the at least one bone anchor.

2. The minimally invasive spinal annulus fibrosus repairing device according to claim 1, wherein when an amount of the bone anchor is one, an amount of the annulus fibrosus anchor is at least two, when the amount of the annulus fibrosus anchor is one, the amount of the bone anchor is at least two, the at least one bone anchor and the at least one annulus fibrosus anchor define a repairing region, and the repairing region has an area larger than that of a tear of the annulus fibrosus of the intervertebral disc.

3. The minimally invasive spinal annulus fibrosus repairing device according to claim 2, wherein the amount of the at least one bone anchor is two, and the amount of the at least one annulus fibrosus anchor is two.

4. The minimally invasive spinal annulus fibrosus repairing device according to claim 1, wherein the at least one suture forms a pulling direction after exiting from the at least one suture outlet hole, and an included angle of 90 degrees is formed between the pulling direction and an extension direction that is far away from and perpendicular to a tear of the annulus fibrosus of the intervertebral disc.

5. The minimally invasive spinal annulus fibrosus repairing device according to claim 1, wherein the at least one bone anchor has a length of 10-20 mm and a width of 3-5 mm.

6. The minimally invasive spinal annulus fibrosus repairing device according to claim 1, wherein the at least one annulus fibrosus anchor has a length of 3-6 mm and a width of 1-3 mm.

7. The minimally invasive spinal annulus fibrosus repairing device according to claim 1, wherein an amount of the suture inlet holes is two.

8. The minimally invasive spinal annulus fibrosus repairing device according to claim 1, wherein the suture penetrating tunnel includes a first tunnel portion and a second tunnel portion opposite to the first tunnel portion, and an included angle of 30-90 degrees is formed between an extension line of the first tunnel portion and an extension line of the second tunnel portion.

9. The minimally invasive spinal annulus fibrosus repairing device according to claim 1, wherein the at least one annulus fibrosus anchor has a columnar structure.

10. The minimally invasive spinal annulus fibrosus repairing device according to claim 9, wherein the at least one annulus fibrosus anchor has a cylindrical structure.

11. The minimally invasive spinal annulus fibrosus repairing device according to claim 1, wherein the at least one annulus fibrosus anchor has a material made of artificial fiber, heterogeneous fiber, titanium alloy, cobalt-based alloy, ceramic, stainless steel, polyetheretherketone (PEEK) resin, or artificial bone.

12. The minimally invasive spinal annulus fibrosus repairing device according to claim 1, wherein the first end and the second end of the at least one suture pass through the suture penetrating tunnel and exit from the at least one suture outlet hole, and the first end and the second end of the at least one suture fixed on the suture fixing portion of the at least one bone anchor are drawn to form a bundle shape in order to seal a tear of the fibrous annulus of the intervertebral disc.

13. The minimally invasive spinal annulus fibrosus repairing device according to claim 1, wherein the body of the at least one bone anchor has a cross-sectional shape of polygon or sheet.

14. The minimally invasive spinal annulus fibrosus repairing device according to claim 1, wherein the suture fixing portion of the body of the at least one bone anchor is a loop.

15. The minimally invasive spinal annulus fibrosus repairing device according to claim 1, wherein the body of the at least one bone anchor further includes a solid frame, and the porous structure is concentrated inside the solid frame and presented in a center of the solid frame.

16. The minimally invasive spinal annulus fibrosus repairing device according to claim 1, wherein the at least one bone anchor has a shape of a porous anchor, a quadrangular prismatic anchor, a threaded anchor or a nail.

17. A method of using the minimally invasive spinal annulus fibrosus repairing device according to claim 1, comprising the following steps:
fixing the at least one bone anchor on the vertebral bone of the annulus fibrous adjacent to the intervertebral disc, wherein the needle tip is embedded in the vertebral bone, and the suture fixing portion is exposed out of the vertebral bone;
threading the first end and the second end of the at least one suture through the at least two suture inlet holes of the at least one annulus fibrosus anchor, and passing through the suture penetrating tunnel to exit from the at least one suture outlet hole;
implanting the at least one annulus fibrosus anchor with the at least one suture into the annulus fibrosus of the intervertebral disc, and pulling the first end and the second end of the at least one suture out of the annulus fibrosus of the intervertebral disc; and
spanning the at least one suture that is pulled out of the annulus fibrosus of the intervertebral disc over a tear of the annulus fibrosus of the intervertebral disc, wherein the first end and the second end of the at least one suture are fixed to the suture fixing portion of the at least one bone anchor.
